(19) United States
(12) Patent Application Publication
(10) Pub. No.: US 2013/0330797 A1
(43) Pub. Date: Dec. 12, 2013

(54) Process for Producing Biogas from Pectin and Lignocellulose Containing Material

(71) Applicant: Hans S. Olsen, Holte (DK)

(72) Inventor: Hans S. Olsen, Holte (DK)

(73) Assignee: Novozymes A/S, Bagsvaerd (DK)

(21) Appl. No.: 13/993,916

(22) PCT Filed: Dec. 22, 2011
(86) PCT No.: PCT/EP2011/073840

§ 371 Date: Aug. 27, 2013

(30) Foreign Application Priority Data
Jan. 4, 2011 (EP) 11150093.0

(51) Int. Cl. C12P 5/02

(52) U.S. Cl. CPC C12P 5/023
USPC 435/167

(57) ABSTRACT

The present invention relates to biogas production processes with enzymatic pre-treatment, said processes comprising the steps of providing a slurry comprising a lignocellulose- and pectin-containing material, water and two or more enzyme treatments; allowing the two or more enzyme-treatment steps to degrade the lignocellulose- and pectin-containing material, and adding the degraded material to a biogas digester tank at a suitable rate and ratio to effectively convert the material to biogas in the digester.

### FIELD OF THE INVENTION

The present invention relates to biogas production processes with enzymatic pre-treatment, said processes comprising the steps of providing a slurry comprising a lignocellulose- and pectin-containing material, water and two or more enzyme treatments; allowing the two or more enzyme-treatment steps to degrade the lignocellulose- and pectin-containing material, and adding the degraded material to a biogas digester tank at a suitable rate and ratio to effectively convert the material to biogas in the digester.

### BACKGROUND OF THE INVENTION

Most natural plant based material comprises a significant amount of lignocellulosic and pectinaceous fibers that are indigestible or only slowly digestible in many biological systems. This has the consequence that a significant fraction of the material will not be digested or only digested in a low degree.

For example, in a usual biogas production plant biomass is fermented under anaerobic conditions to form biogas and a waste material consisting, to a large extent, of lignocellulosic and pectinaceous fibers that are hardly digested at all.

Producing fermentation products, such as, ethanol, from lignocellulose is known in the art and generally includes pre-treating, hydrolyzing and fermenting the material. Lignocellulose-containing feed stock can be hydrolyzed to release fermentable sugars (WO 2010/000858).

The structure of lignocellulose is not directly accessible to enzymatic hydrolysis. Therefore, the lignocellulose is pre-treated in order to break the lignin seal and disrupt the crystalline structure of cellulose. This may cause solubilization and saccharification of the hemicellulose fraction. The cellulose fraction is then hydrolyzed enzymatically, e.g. by cellulolytic enzymes, which degrades the carbohydrate polymers into fermentable sugars.

Current processes for producing biogas from biomass are not yet optimized to achieve the full theoretic conversion to biogas, a fibrous lignocellulosic and pectinaceous waste-material remains which is not converted at all.

### SUMMARY OF THE INVENTION

The invention relates to a biogas production process comprising at least two separate enzymatic pre-treatment steps, where a liquefaction and subsequently a saccharification step is performed of lignocellulosic and pectin containing biomass raw material.

When there is an abundance of pectin in the material, demethylation of the pectin occurs naturally, which over time can result in a drop in pH to acidic conditions, as low as about pH 6. However, many of the enzyme activities suitable for pre-treatment of lignocellulosic biomass material are more effective at neutral to basic pH values. Therefore, it may be necessary to adjust pH up after the liquefaction step and before the saccharification step.

Accordingly, after a drop in pH-value to acidic conditions due to degradation of pectin in the substrate, pH is adjusted to neutral or basic conditions before cell wall degrading enzymes are added that are mainly active above pH 7.

Suitable enzymes for substrates containing pectin are, e.g., pectate lyase (EC 4.2.2.2), an enzyme which degrades pectin by beta-elimination and consequently also lowers the viscosity or pectin methylesterase (EC 3.1.1.11) which hydrolyses pectin.

During the enzymatic degradation steps the polysaccharides like starch, pectin, hemicelluloses, mannan and cellulose is solubilised and converted to mainly oligosaccharides. The protein is hydrolyzed to mainly peptides. The cellulose is converted to cellodextrins.

From the pre-treatment tank(s) the enzyme-treated material is fed to a biogas digester tank in a rate and ratio that fits with the conversion rate to gas.

Before or during the pre-treatment, a milling of the biomass may be done, preferably a wet milling and/or wet grinding, optionally facilitated by addition of the enzymes according to the invention. Temperature and starting pH for the first enzymatic reaction are adjusted to allow the enzymes to function and may be adjusted during the enzymatic reaction steps.

The biomass material can be prewashed with a base, such as, caustic, lime or soda.

Several advantages are provided by the process of the invention, including but not limited to:
Higher conversion rate in the biogas digester tank.
Higher productivity per unit of volume in the digester tank.
Lower investment in tank capacity.
Higher gas production per tank volume.
More efficient conversion of the lignocellulosic pectinaceous material at higher dry matter concentration.
Reduced amounts of unconverted material in the purge.
Higher dry matter content in the unconverted solids.
No need for post-converter or storage tank.
Easier dewatering of unconverted material.
Easier cleaning of the gas phase.

Accordingly, in a first aspect, the invention relates to a biogas production process with two enzymatic pre-treatments, said process comprising the steps of:
(a) providing a slurry comprising a lignocellulose- and pectin-containing material, water and one or more enzyme (FIG. 1; enzyme 1), including, at least one pectate lyase, pectin lyase and/or pectin methylesterase;
(b) allowing the one or more enzyme to catalyze the degradation of the material at a suitable temperature and a starting pH, wherein the pH drops to below 7 over time; and
(c) adding one or more additional enzyme (FIG. 1; enzyme 2) and allowing the one or more additional enzyme to catalyze the degradation of the material further at a suitable temperature and pH; and
(d) adding the enzyme-degraded material to a biogas digester tank at a suitable rate and ratio to effectively convert the material to biogas in the digester.

### BRIEF DESCRIPTION OF DRAWINGS

FIG. 1 shows a schematic outline of the biogas production process principle of the invention; two enzymatic pre-treatment steps are indicated here as separate tanks with an option for recirculation in each step, as outlined below, but they may well be carried out sequentially in a single tank.

FIG. 2 shows the cumulative biogas production during each cycle in Example 5.

FIG. 3 shows biogas production over 40 hours during 3rd cycle in Example 5.

FIG. 4 shows the results from Example 6 comparing a 1-step process using only acidic enzymes with the results of a 2-step process according to the invention; Δ° Brix versus enzyme total dosage with hydrolysis of wet-milled sugar beet pulp at pH=4.5 (init.)−pH=3.5 (end); T=45° C.; t=24 hours using Viscozyme and Cellic CTec2.

### DETAILED DESCRIPTION OF THE INVENTION

In the first aspect the invention relates to biogas processes comprising two or more enzymatic pre-treatment steps, wherein lignocellulose- and pectin-containing materials are hydrolyzed and/or liquefied/solubilised as well as saccharified, respectively.

### Lignocellulose- and Pectin-Containing Material

The term “lignocellulose- and pectin-containing material” means material primarily consisting of cellulose, hemicellulose, lignin and pectin. Lignocellulose- and pectin-containing material is often referred to as “biomass”. Woody biomass is about 45-50% cellulose, 20-25% hemicellulose and 20-25% lignin. Herbaceous materials have lower cellulose, lower lignin and higher hemicellulose contents.

Cellulose is a linear beta 1->4 linked polymer of glucose. It is the principal component of all higher plant cell walls. In nature cellulose exists in crystalline and amorphous states. The thermodynamic stability of the beta 1->4 linkage and the capacity of cellulose to form internal hydrogen bonds gives it great structural strength. Cellulose is degraded to glucose through hydrolytic cleavage of the glycosidic bond.

Hemicellulose is a term used to refer to a wide variety of heteropolysaccharides found in association with cellulose and lignin in both woody and herbaceous plant species. The sugar composition varies with the plant species, but in angiosperms, the principal hemicellulosic sugar is xylose. Like cellulose, xylose occurs in the beta 1->4 linked backbone of the polymer. In gymnosperms, the principal component sugar is mannose. Arabinose is found as a side branch in some hemicelluloses.

Lignin is a phenylpropane polymer. Unlike cellulose and hemicellulose, lignin cannot be depolymerized by hydrolysis. Cleavage of the principal bonds in lignin requires oxidation.

Pectin is a family of complex polysaccharides that contain 1,4-linked α-D-galactosyluronic acid residues. Three pectic polysaccharides have been isolated from plant primary cell walls and structurally characterized. These are:
Homogalacturonans
Substituted galacturonans
Rhamnogalacturonans

Homogalacturonans are linear chains of α-(1-4)-linked D-galacturonic acid. Substituted galacturonans are characterized by the presence of saccharide appendant residues (such as D-xylose or D-apiose in the respective cases of xylogalacturonan and apiogalacturonan) branching from a backbone of D-galacturonic acid residues. Rhamnogalacturonan I pectins (RG-I) contain a backbone of the repeating disaccharide: 4)-α-D-galacturonic acid-(1,2)-α-L-rhamnose-(1. From many of the rhamnose residues, side chains of various neutral sugars branch off. The neutral sugars are mainly D-galactose, L-arabinose and D-xylose, the types and proportions of neutral sugars varying with the origin of pectin.

Another structural type of pectin is rhamnogalacturonan II (RG-II), which is a less frequent complex, highly branched polysaccharide. Rhamnogalacturonan II is classified by some authors within the group of substituted galacturonans since the rhamnogalacturonan II backbone is made exclusively of D-galacturonic acid units.

In nature, around 80% of carboxyl groups of galacturonic acid are esterified with methanol. This proportion is decreased more or less during pectin extraction. The ratio of esterified to non-esterified galacturonic acid determines the behavior of pectin in food applications. This is why pectins are classified as high- vs. low-ester pectins—or in short HM vs. LM-pectins, with more or less than half of all the galacturonic acid esterified. The non-esterified galacturonic acid units can be either free acids (carboxyl groups) or salts with sodium, potassium or calcium. The salts of partially esterified pectins are called pectinates, if the degree of esterification is below 5% the salts are called pectates, the insoluble acid form, pectic acid.

Some plants like sugar beet, potatoes and pears contain pectins with acetylated galacturonic acid in addition to methyl esters. Acetylation prevents gel-formation but increases the stabilising and emulsifying effects of pectin.

Apples, guavas, quince, plums, gooseberries, oranges and other citrus fruits, contain large amounts of pectin, while soft fruits like cherries, grapes and strawberries contain small amounts of pectin. Typical levels of pectin in plants are (fresh weight):
apples, 1-1.5%
apricot, 1%
cherries, 0.4%
oranges 0.5-3.5%
carrots approx. 1.4%
citrus peels, 30%

The lignocellulose- and pectin-containing material may be any material containing lignocellulose and pectin. In a preferred embodiment the lignocellulose- and pectin-containing material contains at least 30 wt-%, preferably at least 50 wt-%, more preferably at least 70 wt-%, even more preferably at least 90 wt-% lignocellulose. In a preferred embodiment the material contains at least 5 wt-%, preferably at least 10 wt-%, 20 wt-%, 30 wt-%, 40 wt-%, or preferably at least 50 wt-%, 60 wt-%, more preferably at least 70 wt-%, even more preferably at least 80 wt-%, or 90 wt-% pectin. It is to be understood that the material may also comprise other constituents such as proteinaceous material, starchy material, and sugars, such as fermentable sugars and/or un-fermentable sugars.

Lignocellulose- and pectin-containing material is generally found, for example, in the stems, leaves, hulls, husks, and cobs of plants or leaves, branches, and wood of trees. Lignocellulose- and pectin-containing material can also be, but is not limited to, herbaceous material, agricultural residues, forestry residues, municipal solid wastes, waste paper, and pulp and paper mill residues. It is to be understood that lignocellulose- and pectin-containing material may be in the form of plant cell wall material containing lignin, cellulose and hemicellulose in a mixed matrix.

In a preferred embodiment the lignocellulose- and pectin-containing material comprises or is derived from potato pulp, sweet potato pulp, cassava pulp, sugar beet pulp, apple pulp, pear pulp, banana pulp, orange pomace, grape pomace, lemon pulp, pineapple pulp. as well as waste residue from carrots, cereal straw, wheat straw, palm fronds, palm fruits, empty palm fruit bunches, palm residues, switch grass, miscanthus, rice hulls, municipal solid waste, industrial organic waste, office paper, bagasse of sugar cane or mixtures thereof.

In a preferred embodiment of the first aspect of the invention, the content of lignocellulose- and pectin-containing material in the slurry is adjusted by continuous or stepwise addition of said material to the slurry during step (b) and/or step (c).

### Pre-Treatment

The lignocellulose- and pectin-containing material may be pre-treated in any suitable way. The pre-treatment is carried out before or at the same time as the enzymatic hydrolysis. The goal of pre-treatment is to reduce the particle size, separate and/or release cellulose; hemicellulose and/or lignin and in this way increase the rate of hydrolysis. Pre-treatment processes such as wet-oxidation and alkaline pre-treatment targets lignin, while dilute acid and auto-hydrolysis targets hemicellulose. Steam explosion is an example of a pre-treatment that targets lignin.

The pre-treatment step may be a conventional pre-treatment step using techniques well known in the art. In a preferred embodiment pre-treatment takes place in a slurry of lignocellulose- and pectin-containing material and water. The material may be present during pre-treatment in an amount between 10-80 wt-%, preferably between 20-70 wt-%, especially between 30-60 wt-%, such as around 50 wt-%.

It is preferred that step (b) and/or (c) of the first aspect of the invention is carried out at a temperature in the range from 20-70° C., preferably 30-60° C., and more preferably 40-50° C.

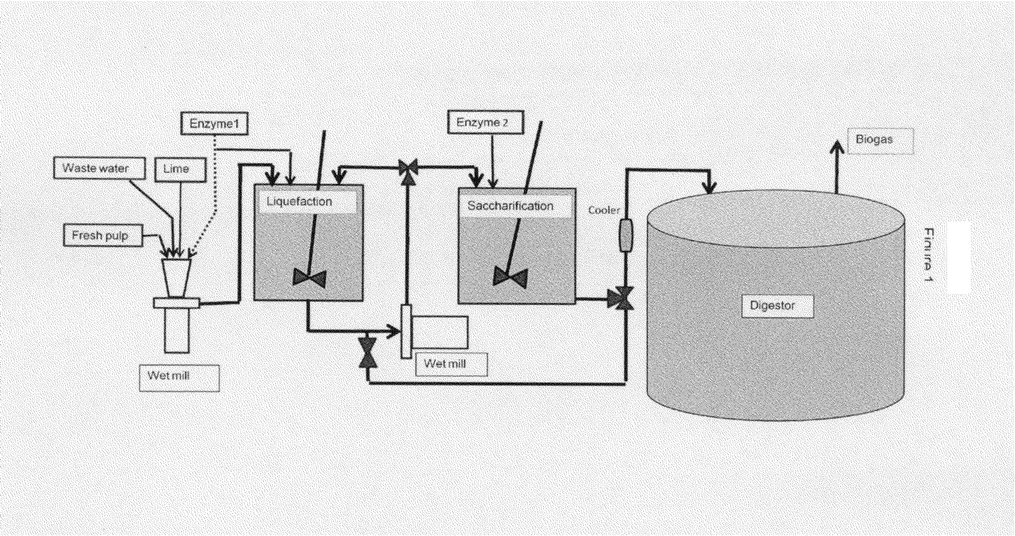

In a preferred embodiment of the first aspect of the invention, a solids separation step is performed after step (b) but before step (c) to purge not-solubilized solids (FIG. 1) and optionally feed them back into step (a) of the process.

In another preferred embodiment of the first aspect, a solids separation step is performed after step (c) but before step (d) to purge not-solubilized solids (FIG. 1) and optionally feed them back into step (a) or (c) of the process.

Chemical, Mechanical and/or Biological Pre-Treatment

The lignocellulose- and pectin-containing material may according to the invention be chemically, mechanically and/or biologically pre-treated before the first enzyme hydrolysis in accordance with the process of the invention. Mechanical pre-treatment (often referred to as “physical”—pre-treatment) may be carried out alone or may be combined with other pre-treatment processes.

Preferably, the chemical, mechanical and/or biological pre-treatment is carried out prior to the first enzyme treatment step. Alternatively, the chemical, mechanical and/or biological pre-treatment may be carried out simultaneously with the first enzyme treatment step, such as simultaneously with addition of one or more enzymes, and/or other enzyme activities.

### Chemical Pre-Treatment

The term “chemical pre-treatment” refers to any chemical pre-treatment which promotes the separation and/or release of cellulose, hemicellulose, pectin and/or lignin. Examples of suitable chemical pre-treatments include treatment with; for example, dilute acid, lime, alkaline, organic solvent, ammonia, sulfur dioxide, carbon dioxide. Further, wet oxidation and pH-controlled hydrothermolysis are also considered chemical pre-treatment.

Other pre-treatment techniques are also contemplated according to the invention. Cellulose solvent treatment has been shown to convert about 90% of cellulose to glucose. It has also been shown that enzymatic hydrolysis could be greatly enhanced when the lignocellulose structure is disrupted. Alkaline H₂O₂, ozone, organosolv (uses Lewis acids, FeCl₃, Al₂(SO₄)₃ (in aqueous alcohols), glycerol, dioxane, phenol, or ethylene glycol are among solvents known to disrupt cellulose structure and promote hydrolysis (Mosier et al. Bioresource Technology 96 (2005), p. 673-686).

Alkaline chemical pre-treatment with base, e.g., NaOH, Na₂CO₃, NaHCO₃, Ca(OH)₂, lime hydrate, ammonia and/or KOH or the like, is also within the scope of the invention. Pre-treatment processes using ammonia are described in, e.g., WO 2006/110891, WO 2006/11899, WO 2006/11900, WO 2006/110901, which are hereby incorporated by reference. Also the Kraft pulping process as described for example in “Pulp Processes” by Sven A. Rydholm, page 583-648. ISBN 0-89874-856-9 (1985) might be used. The solid pulp (about 50% by weight based on the dry wood chips) is collected and washed before the enzymatic treatments.

Wet oxidation techniques involve use of oxidizing agents, such as: sulfite based oxidizing agents or the like. Examples of solvent pre-treatments include treatment with DMSO (Dimethyl Sulfoxide) or the like. Chemical pre-treatment is generally carried out for 1 to 60 minutes, such as from 5 to 30 minutes, but may be carried out for shorter or longer periods of time dependent on the material to be pre-treated.

Other examples of suitable pre-treatment processes are described by Schell et al. (2003) Appl. Biochem. and Biotechn. Vol. 105-108, p. 69-85, and Mosier et al. Bioresource Technology 96 (2005) 673-686, and US publication no. 2002/0164730, which references are hereby all incorporated by reference.

### Mechanical Pre-Treatment

The term “mechanical pre-treatment” refers to any mechanical (or physical) pre-treatment which promotes the separation and/or release of cellulose, hemicellulose and/or lignin from lignocellulose- and pectin-containing material. For example, mechanical pre-treatment includes various types of milling, irradiation, steaming/steam explosion, and hydrothermolysis.

Mechanical pre-treatment includes comminution (mechanical reduction of the size). Comminution includes dry milling, wet milling and vibratory ball milling. Mechanical pre-treatment may involve high pressure and/or high temperature (steam explosion). In an embodiment of the invention high pressure means pressure in the range from 300 to 600 psi, preferably 400 to 500 psi, such as around 450 psi. In an embodiment of the invention high temperature means temperatures in the range from about 100 to 300° C., preferably from about 140 to 235° C. In a preferred embodiment mechanical pre-treatment is carried out as a batch-process, in a steam gun hydrolyzer system which uses high pressure and high temperature as defined above. A Sunds Hydrolyzer (available from Sunds Defibrator AB (Sweden) may be used for this.

For wet milling a colloidal mill type MZ from Romaco FrymaKoruma, GmbH, Neuenburg Germany has been found useful. Furthermore a combined shredder and colloidal mill type Fine Gorator from Hoelschertechnic-Gorator® GmbH & Co. Gescher, Germany was found useful for making a particle size less than 300 micrometer.

In a preferred embodiment the lignocellulose- and pectin-containing material is subjected to a irradiation pre-treatment. The term “irradiation pre-treatment” refers to any pre-treatment by microwave e.g. as described by Zhu et al. “Production of ethanol from microwave-assisted alkali pre-treated wheat straw” in Process Biochemistry 41 (2006) 869-873 or ultrasonic pre-treatment, e.g., as described by e.g. Li et al. “A kinetic study on enzymatic hydrolysis of a variety of pulps for its enhancement with continuous ultrasonic irradiation”, in Biochemical Engineering Journal 19 (2004) 155-164.

In another preferred embodiment, the lignocellulose- and pectin-containing material or the slurry is homogenized; preferably by milling, wet-milling, grinding or wet-grinding prior to or during step (b) and/or step (c) of the first aspect.

### Combined Chemical and Mechanical Pre-Treatment

In a preferred embodiment the lignocellulose- and pectin-containing material is subjected to both chemical and mechanical pre-treatment. For instance, the pre-treatment step may involve dilute or mild acid treatment and high temperature and/or pressure treatment. The chemical and mechanical pre-treatments may be carried out sequentially or simultaneously, as desired.

In a preferred embodiment the pre-treatment is carried out as a dilute and/or mild acid steam explosion step. In another preferred embodiment pre-treatment is carried out as an ammonia fiber explosion step (or AFEX pre-treatment step).

In yet another preferred embodiment, a base is added to the lignocellulose- and pectin-containing material or the slurry prior to or while it is being homogenized; preferably the base is NaOH, Na₂CO₃, NaHCO₃, Ca(OH)₂, lime hydrate, ammonia and/or KOH.

### Biological Pre-Treatment

The term “biological pre-treatment” refers to any biological pre-treatment which promotes the separation and/or release of cellulose, hemicellulose, and/or lignin from the lignocellulose- and pectin-containing material. Known biological pre-treatment techniques involve applying lignin-solubilizing microorganisms (see, for example, Hsu, T.-A., 1996, Pretreatment of biomass, in Handbook on Bioethanol: Production and Utilization, Wyman, C. E., ed., Taylor & Francis, Washington, D.C., 179-212; Ghosh, P., and Singh, A., 1993, Physicochemical and biological treatments for enzymatic/microbial conversion of lignocellulosic biomass, Adv. Appl. Microbiol. 39: 295-333; McMillan, J. D., 1994, Pretreating lignocellulosic biomass: a review, in Enzymatic Conversion of Biomass for Fuels Production, Himmel, M. E., Baker, J. 0., and Overend, R. P., eds., ACS Symposium Series 566, American Chemical Society, Washington, D.C., chapter 15; Gong, C. S., Cao, N.J., Du, J., and Tsao, G. T., 1999, Ethanol production from renewable resources, in Advances in Biochemical Engineering/Biotechnology, Scheper, T., ed., Springer-Verlag Berlin Heidelberg, Germany, 65: 207-241; Olsson, L., and Hahn-Hagerdal, B., 1996, Fermentation of lignocellulosic hydrolysates for ethanol production, Enz. Microb. Tech. 18: 312-331; and Vallander, L., and Eriksson, K.-E. L., 1990, Production of ethanol from lignocellulosic materials: State of the art, Adv. Biochem. Eng./Biotechnol. 42: 63-95).

### Enzymatic Hydrolysis

Before the pre-treated lignocellulose- and pectin-containing material is fermented it is hydrolyzed enzymatically to break down especially hemicellulose and/or cellulose into fermentable sugars.

According to the invention the enzymatic hydrolysis is performed in several steps. The lignocellulose- and pectin-containing material to be hydrolyzed constitutes above 2.5 wt-% DM, preferably above 5 wt-% DM, preferably above 10 wt-% DM, preferably above 15 wt-% DM, preferably above 20 wt.-% DM, more preferably above 25 wt-% DM of the slurry of step a).

In step (b) of the invention, the lignocellulose- and pectin-containing material is subjected to the action of one, or several or all enzyme activities selected from the group consisting of an amylolytic enzyme, a lipolytic enzyme, a proteolytic enzyme, a cellulolytic enzyme, an oxidoreductase and a plant cell-wall degrading enzyme.

In a preferred embodiment, the one or more enzyme or additional enzyme is selected from the group consisting of an amylolytic enzyme, a lipolytic enzyme, a proteolytic enzyme, a cellulolytic enzyme, an oxidoreductase and a plant cell-wall degrading enzymeone; preferably the one or more enzyme or additional enzyme is selected from the group consisting of aminopeptidase, alpha-amylase, amyloglucosidase, arabinofuranosidase, arabinoxylanase, beta-glucanase, carbohydrase, carboxypeptidase, catalase, cellobiohydrolase, cellulase, chitinase, cutinase, cyclodextrin glycosyltransferase, ferulic acid esterase, deoxyribonuclease, endo-cellulase, endo-glucanase, endo-xylanase, esterase, galactosidase, beta-galactosidase, glucoamylase, glucose oxidase, glucosidase, haloperoxidase, hemicellulase, invertase, isomerase, laccase, ligase, lipase, lyase, mannanase, mannosidase, oxidase, pectate lyase, pectin lyase, pectin trans-eliminase, pectin ethylesterase, pectin methylesterase, pectinolytic enzyme, peroxidase, protease, phytase, phenoloxidase, polygalacturonase, polyphenoloxidase, proteolytic enzyme, rhamnogalacturonan lyase, rhamnoglucanase, rhamnogalacturonase, ribonuclease, SPS-ase, transferase, transglutaminase, xylanase and xyloglucanase.

In another preferred embodiment, the one or more enzyme is a protease, a pectate lyase, a ferulic acid esterase and/or a mannanase.

It is preferred that the starting pH in step (b) of the first embodiment is between 7 and 12, such as from 7.6 to 10; preferably from 8 to 10, or from 8 to 9, preferably around pH 8.5. As already mentioned, when there is an abundance of pectin in the material, demethylation of the pectin occurs naturally, which over time can result in a drop in pH to acidic conditions as low as about pH 6, before step (c) is begun.

In yet another preferred embodiment, step (c) of the first aspect is carried out at a pH in the range from 3 to 7; preferably from 4 to 6; most preferably at a pH value around 5.

It is noteworthy, that the pre-treated biomass material should preferably have a neutral to basic pH value when it is added to the biogas digester; it has been suggested that addition of acidic biomass halt the biogas conversion process due to a proposed inhibition of the common methanogenic microorganisms.

### Enzymes

Even if not specifically mentioned in context of a process or process of the invention, it is to be understood that the enzyme(s) (as well as other compounds) are used in an “effective amount”

### Proteases

Any protease suitable for use under alkaline conditions can be used. Suitable proteases include those of animal, vegetable or microbial origin. Microbial origin is preferred. Chemically or genetically modified mutants are included. The protease may be a serine protease, preferably an alkaline microbial protease or a trypsin-like protease. Examples of alkaline proteases are subtilisins, especially those derived from Bacillus, e.g., subtilisin Novo, subtilisin Carlsberg, subtilisin 309, subtilisin 147 and subtilisin 168 (described in WO 89/06279). Examples of trypsin-like proteases are trypsin (e.g. of porcine or bovine origin) and the Fusarium protease described in WO 89/06270.

Preferred commercially available protease enzymes include those sold under the trade names Everlase™, Kannase™, Alcalase™, Savinase™, Primase™, Durazym™, and Esperase™ by Novozymes A/S (Denmark), those sold under the tradename Maxatase, Maxacal, Maxapem, Properase, Purafect and Purafect OXP by Genencor International, and those sold under the tradename Opticlean and Optimase by Solvay Enzymes.

### Hemicellulolytic Enzymes

Any hemicellulase suitable for use in hydrolyzing hemicellulose may be used. Preferred hemicellulases include pectate lyases, xylanases, arabinofuranosidases, acetyl xylan esterase, ferulic acid esterase, glucuronidases, endo-galactanase, mannases, endo or exo arabinases, exo-galactanses, and mixtures of two or more thereof. Preferably, the hemicellulase for use in the present invention is an endo-acting hemicellulase, and more preferably, the hemicellulase is an endo-acting hemicellulase.

In an embodiment the hemicellulase is a xylanase. In an embodiment the xylanase may preferably be of microbial origin, such as of fungal origin (e.g., Trichoderma, Meripilus, Humicola, Aspergillus, Fusarium) or from a bacterium (e.g., Bacillus). In a preferred embodiment the xylanase is derived from a filamentous fungus, preferably derived from a strain of Aspergillus, such as Aspergillus aculeatus; or a strain of Humicola, preferably Humicola lanuginosa. The xylanase may preferably be an endo-1,4-beta-xylanase, more preferably an endo-1,4-beta-xylanase of GH10 or GH11. Examples of commercial xylanases include SHEARZYME® 200L, SHEARZYME® 500L, BIOFEED WHEAT®, and PULPZYME™ HC derived from Bacillus agaradhaerens (from Novozymes) and GC 880, SPEZYME® CP (from Genencor Int).

The hemicellulase may be added in an amount effective to hydrolyze hemicellulose, such as, in amounts from about 0.001 to 0.5 wt-% of total dry matter (DM), more preferably from about 0.05 to 0.5 wt-% of DM.

Xylanases may be added in the amounts of 1.0-1000 FXU/kg DM, preferably from 5-500 FXU/kg DM, preferably from 5-100 FXU/kg DM and most preferably from 10-100 FXU/kg DM.

Xylanases may alternatively be added in amounts of 0.001-1.0 g/kg DM substrate, preferably in the amounts of 0.005-0.5 g/kg DM substrate, and most preferably from 0.05-0.10 g/kg DM substrate.

### Pectolytic Enzymes (or Pectinases)

Any pectinolytic enzyme that can degrade the pectin composition of plant cell walls may be used in practicing the present invention. Suitable pectinases include, without limitation, those of fungal or bacterial origin. Chemically or genetically modified pectinases are also encompassed. Preferably, the pectinase used in the invention are recombinantly produced and are mono-component enzymes.

Pectinases can be classified according to their preferential substrate, highly methyl-esterified pectin or low methyl-esterified pectin and polygalacturonic acid (pectate), and their reaction mechanism, beta-elimination or hydrolysis. Pectinases can be mainly endo-acting, cutting the polymer at random sites within the chain to give a mixture of oligomers, or they may be exo-acting, attacking from one end of the polymer and producing monomers or dimers. Several pectinase activities acting on the smooth regions of pectin are included in the classification of enzymes provided by Enzyme Nomenclature (1992), e.g., pectate lyase (EC 4.2.2.2), pectin lyase (EC 4.2.2.10), polygalacturonase (EC 3.2.1.15), exo-polygalacturonase (EC 3.2.1.67), exo-polygalacturonate lyase (EC 4.2.2.9) and exo-poly-alpha-galacturonosidase (EC 3.2.1.82).

In embodiments the pectinase is a pectate lyase. Pectate lyase enzymatic activity as used herein refers to catalysis of the random cleavage of alpha-1,4-glycosidic linkages in pectic acid (also called polygalcturonic acid) by transelimination. Pectate lyases are also termed polygalacturonate lyases and poly(1,4-α-D-galacturonide) lyases.

The Pectate lyase (EC 4.2.2.2) is an enzyme which catalyse the random cleavage of α-1,4-glycosidic linkages in pectic acid (also called polygalacturonic acid) by transelimination. Pectate lyases also include polygalacturonate lyases and poly(1,4-α-D-galacturonide) lyases.

Examples of preferred pectate lyases are those that have been cloned from different bacterial genera such as Erwinia, Pseudomonas, Klebsiella, Xanthomonas and Bacillus, especially Bacillus licheniformis (U.S. Pat. No. 6,124,127), as well as from Bacillus subtilis (Nasser et al. (1993) FEBS Letts. 335:319-326) and Bacillus sp. YA-14 (Kim et al. (1994) Biosci. Biotech. Biochem. 58:947-949). Purification of pectate lyases with maximum activity in the pH range of 8-10 produced by Bacillus pumilus (Dave and Vaughn (1971) J. Bacteriol. 108:166-174), B. polymyxa (Nagel and Vaughn (1961) Arch. Biochem. Biophys. 93:344-352), B. stearothermophilus (Karbassi and Vaughn (1980) Can. J. Microbiol. 26:377-384), Bacillus sp. (Hasegawa and Nagel (1966) J. Food Sci. 31:838-845) and Bacillus sp. RK9 (Kelly and Fogarty (1978) Can. J. Microbiol. 24:1164-1172) have also been described.

A preferred pectate lyase may be obtained from Bacillus licheniformis as described in U.S. Pat. No. 6,124,127.

Other pectate lyases could be those that comprise the amino acid sequence of a pectate lyase disclosed in Heffron et al., (1995) Mol. Plant-Microbe Interact. 8: 331-334 and Henrissat et al., (1995) Plant Physiol. 107: 963-976.

A single enzyme or a combination of pectate lyases may be used. A preferred commercial pectate lyase preparation suitable for the invention is BioPrep® 3000 L available from Novozymes A/S.

### Mannanases

In the context of the present invention a mannanase is a beta-mannanase and defined as an enzyme belonging to EC 3.2.1.78.

Mannanases have been identified in several Bacillus organisms. For example, Talbot et al., Appl. Environ. Microbiol., Vol. 56, No. 11, pp. 3505-3510 (1990) describes a beta-mannanase derived from Bacillus stearothermophilus having an optimum pH of 5.5-7.5. Mendoza et al., World J. Microbiol. Biotech., Vol. 10, No. 5, pp. 551-555 (1994) describes a beta-mannanase derived from Bacillus subtilis having an optimum activity at pH 5.0 and 55° C. JP-03047076 discloses a beta-mannanase derived from Bacillus sp., having an optimum pH of 8-10. JP-63056289 describes the production of an alkaline, thermostable beta-mannanase. JP-08051975 discloses alkaline beta-mannanases from alkalophilic Bacillus sp. AM-001. A purified mannanase from Bacillus amyloliquefaciens is disclosed in WO 97/11164. WO 94/25576 discloses an enzyme from Aspergillus aculeatus, CBS 101.43, exhibiting mannanase activity and WO 93/24622 discloses a mannanase isolated from Trichoderma reesei.

The mannanase may be derived from a strain of the genus Bacillus, such as the amino acid sequence having the sequence deposited as GENESEQP accession number AAY54122 or an amino acid sequence which is homologous to this amino acid sequence. A suitable commercial mannanase preparation is Mannaway® produced by Novozymes A/S.

### Ferulic Esterases

In the context of the present invention a ferulic esterase is defined as an enzyme belonging to EC 3.1.1.73.

A suitable ferulic esterase preparation can be obtained from Malabrancea, e.g., from P. cinnamomea, such as e.g. a preparation comprising the ferulic esterase having the amino acid sequence shown in SEQ ID NO:2 in European patent application number 07121322.7, or an amino acid sequence which is homologous to this amino acid sequence.

Another suitable ferulic esterase preparation can be obtained from Penicillium, e.g., from P. aurantiogriseum, such as e.g. a preparation comprising the ferulic esterase having the amino acid sequence shown in SEQ ID NO:2 in European patent application number 0815469.7, or an amino acid sequence which is homologous to this amino acid sequence. A suitable commercial ferulic esterase preparation is NOVOZYM® 342 L produced by Novozymes A/S.

### Alkaline Endo-Glucanases

The term “endoglucanase” means an endo-1,4-(1,3;1,4)-beta-D-glucan 4-glucanohydrolase (E.C. No. 3.2.1.4), which catalyses endo-hydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3 glucans such as cereal beta-D-glucans or xyloglucans, and other plant material containing cellulosic components. Alkaline endo-glucanases are endo-glucanases having activity under alkaline conditions.

In a preferred embodiment endoglucanases may be derived from a strain of the genus Trichoderma, preferably a strain of Trichoderma reesei; a strain of the genus Humicola, such as a strain of Humicola insolens; or a strain of Chrysosporium, preferably a strain of Chrysosporium lucknowense.

In a preferred embodiment endoglucanases may be derived from a strain of the genus Bacillus akibai.

In an embodiment the alkaline endo-glucanase composition is one of the commercially available products CAREZYME®, ENDOLASE® and CELLUCLEAN® (Novozymes A/S, Denmark). The enzyme may be applied in a dosage of 1-100 g/kg cellulose.

### Acid Cellulolytic Activity

The term “acid cellulolytic activity” as used herein are understood as comprising enzymes having cellobiohydrolase activity (EC 3.2.1.91), e.g., cellobiohydrolase I and/or cellobiohydrolase II, as well as endo-glucanase activity (EC 3.2.1.4) and/or beta-glucosidase activity (EC 3.2.1.21) having activity at pH below 6.

The cellulolytic activity may, in a preferred embodiment, be in the form of a preparation of enzymes of fungal origin, such as from a strain of the genus Trichoderma, preferably a strain of Trichoderma reesei; a strain of the genus Humicola, such as a strain of Humicola insolens; or a strain of Chrysosporium, preferably a strain of Chrysosporium lucknowense.

In preferred embodiment the cellulolytic enzyme preparation contains one or more of the following activities: endoglucanase, cellobiohydrolases I and II, and beta-glucosidase activity.

In a preferred embodiment cellulolytic enzyme preparation is a composition disclosed in WO2008/151079, which is hereby incorporated by reference. In a preferred embodiment the cellulolytic enzyme preparation comprising a polypeptide having cellulolytic enhancing activity, preferably a family GH61A polypeptide, preferably those disclosed in WO 2005/074656 (Novozymes). The cellulolytic enzyme preparation may further comprise beta-glucosidase, such as beta-glucosidase derived from a strain of the genus Trichoderma, Aspergillus or Penicillium, including the fusion protein having beta-glucosidase activity disclosed in co-pending application U.S. 60/832,511 (Novozymes). In a preferred embodiment the cellulolytic enzyme preparation may also comprises a CBH II enzyme, preferably Thielavia terrestris cellobiohydrolase II (CEL6A). In another preferred embodiment the cellulolytic enzyme preparation may also comprise cellulolytic enzymes; preferably those derived from Trichoderma reesei or Humicola insolens.

The cellulolytic enzyme composition may also comprising a polypeptide having cellulolytic enhancing activity (GH61A) disclosed in WO 2005/074656; a beta-glucosidase (e.g., fusion protein disclosed in U.S. 60/832,511 and PCT/US2007/074038), and cellulolytic enzymes derived from Trichoderma reesei.

In another preferred embodiment the cellulolytic composition comprising a polypeptide having cellulolytic enhancing activity (GH61A) disclosed in WO 2005/074656; a beta-glucosidase (e.g., fusion protein disclosed in U.S. 60/832,511 and PCT/US2007/074038), Thielavia terrestris cellobiohydrolase II (CEL6A), and cellulolytic enzymes preparation derived from Trichoderma reesei.

In an embodiment the cellulolytic enzyme composition is the commercially available product CELLUCLAST™ 1.5L, CELLUZYME™ (Novozymes A/S, Denmark) or ACCELLARASE™ 1000 (Genencor Int, Inc., USA).

The cellulolytic activity may be dosed in the range from 0.1-100 FPU per gram DM, preferably 0.5-50 FPU per gram DM, especially 1-20 FPU per gram DM.

### Cellulolytic Enhancing Activity

The term “cellulolytic enhancing activity” is defined herein as a biological activity that enhances the hydrolysis of a lignocellulose derived material by proteins having cellulolytic activity. For purposes of the present invention, cellulolytic enhancing activity is determined by measuring the increase in reducing sugars or in the increase of the total of cellobiose and glucose from the hydrolysis of a lignocellulose derived material, e.g., pre-treated lignocellulose- and pectin-containing material by cellulolytic protein under the following conditions: 1-50 mg of total protein/g of cellulose in PCS (pre-treated corn stover), wherein total protein is comprised of 80-99.5% w/w cellulolytic protein/g of cellulose in PCS and 0.5-20% w/w protein of cellulolytic enhancing activity for 1-7 day at 50° C. compared to a control hydrolysis with equal total protein loading without cellulolytic enhancing activity (1-50 mg of cellulolytic protein/g of cellulose in PCS).

The polypeptides having cellulolytic enhancing activity enhance the hydrolysis of a lignocellulose derived material catalyzed by proteins having cellulolytic activity by reducing the amount of cellulolytic enzyme required to reach the same degree of hydrolysis preferably at least 0.1-fold, more at least 0.2-fold, more preferably at least 0.3-fold, more preferably at least 0.4-fold, more preferably at least 0.5-fold, more preferably at least 1-fold, more preferably at least 3-fold, more preferably at least 4-fold, more preferably at least 5-fold, more preferably at least 10-fold, more preferably at least 20-fold, even more preferably at least 30-fold, most preferably at least 50-fold, and even most preferably at least 100-fold.

In a preferred embodiment the hydrolysis and/or fermentation is carried out in the presence of a cellulolytic enzyme in combination with a polypeptide having enhancing activity. In a preferred embodiment the polypeptide having enhancing activity is a family GH61A polypeptide. WO 2005/074647 discloses isolated polypeptides having cellulolytic enhancing activity and polynucleotides thereof from Thielavia terrestris. WO 2005/074656 discloses an isolated polypeptide having cellulolytic enhancing activity and a polynucleotide thereof from Thermoascus aurantiacus. U.S. Published Application Serial No. 2007/0077630 discloses an isolated polypeptide having cellulolytic enhancing activity and a polynucleotide thereof from Trichoderma reesei.

### Alpha-Amylase

According to the invention any alpha-amylase may be used, such as of fungal, bacterial or plant origin. In a preferred embodiment the alpha-amylase is an acid alpha-amylase, e.g., acid fungal alpha-amylase or acid bacterial alpha-amylase. The term “acid alpha-amylase” means an alpha-amylase (E.C. 3.2.1.1) which added in an effective amount has activity optimum at a pH in the range of 3 to 7, preferably from 3.5 to 6, or more preferably from 4-5.

### Bacterial Alpha-Amylase

According to the invention a bacterial alpha-amylase is preferably derived from the genus Bacillus.

In a preferred embodiment the Bacillus alpha-amylase is derived from a strain of Bacillus licheniformis, Bacillus amyloliquefaciens, Bacillus subtilis or Bacillus stearothermophilus, but may also be derived from other Bacillus sp. Specific examples of contemplated alpha-amylases include the Bacillus licheniformis alpha-amylase shown in SEQ ID NO: 4 in WO 99/19467, the Bacillus amyloliquefaciens alpha-amylase SEQ ID NO: 5 in WO 99/19467 and the Bacillus stearothermophilus alpha-amylase shown in SEQ ID NO: 3 in WO 99/19467 (all sequences hereby incorporated by reference). In an embodiment the alpha-amylase may be an enzyme having a degree of identity of at least 60%, preferably at least 70%, more preferred at least 80%, even more preferred at least 90%, such as at least 95%, at least 96%, at least 97%, at least 98% or at least 99% to any of the sequences shown in SEQ ID NOS: 1, 2 or 3, respectively, in WO 99/19467.

The Bacillus alpha-amylase may also be a variant and/or hybrid, especially one described in any of WO 96/23873, WO 96/23874, WO 97/41213, WO 99/19467, WO 00/60059, and WO 02/10355 (all documents hereby incorporated by reference). Specifically contemplated alpha-amylase variants are disclosed in U.S. Pat. Nos. 6,093,562, 6,297,038 or U.S. Pat. No. 6,187,576 (hereby incorporated by reference) and include Bacillus stearothermophilus alpha-amylase (BSG alpha-amylase) variants having a deletion of one or two amino acid in positions R179 to G182, preferably a double deletion disclosed in WO 1996/023873—see e.g., page 20, lines 1-10 (hereby incorporated by reference), preferably corresponding to delta(181-182) compared to the wild-type BSG alpha-amylase amino acid sequence set forth in SEQ ID NO:3 disclosed in WO 99/19467 or deletion of amino acids R179 and G180 using SEQ ID NO:3 in WO 99/19467 for numbering (which reference is hereby incorporated by reference). Even more preferred are Bacillus alpha-amylases, especially Bacillus stearothermophilus alpha-amylase, which have a double deletion corresponding to delta(181-182) and further comprise a N193F substitution (also denoted 1181*+G182*+N193F) compared to the wild-type BSG alpha-amylase amino acid sequence set forth in SEQ ID NO:3 disclosed in WO 99/19467.

In an embodiment the bacterial alpha-amylase is dosed in an amount of 0.0005-5 KNU per g DM, preferably 0.001-1 KNU per g DM, such as around 0.050 KNU per g DM.

### Fungal Alpha-Amylase

Fungal alpha-amylases include alpha-amylases derived from a strain of the genus Aspergillus, such as, Aspergillus oryzae, Aspergillus niger and Aspergillis kawachii alpha-amylases.

A preferred acidic fungal alpha-amylase is a Fungamyl-like alpha-amylase which is derived from a strain of Aspergillus oryzae. According to the present invention, the term “Fungamyl-like alpha-amylase” indicates an alpha-amylase which exhibits a high identity, i.e. at least 70%, at least 75%, at least 80%, at least 85%, at least 90%, at least 95%, at least 96%, at least 97%, at least 98%, at least 99% or even 100% identity to the mature part of the amino acid sequence shown in SEQ ID NO: 10 in WO 96/23874.

Another preferred acid alpha-amylase is derived from a strain Aspergillus niger. In a preferred embodiment the acid fungal alpha-amylase is the one from Aspergillus niger disclosed as “AMYA_ASPNG” in the Swiss-prot/TeEMBL database under the primary accession no. P56271 and described in WO 89/01969 (Example 3—incorporated by reference). A commercially available acid fungal alpha-amylase derived from Aspergillus niger is SP288 (available from Novozymes A/S, Denmark).

Other contemplated wild-type alpha-amylases include those derived from a strain of the genera Rhizomucor and Meripilus, preferably a strain of Rhizomucor pusillus (WO 2004/055178 incorporated by reference) or Meripilus giganteus.

In a preferred embodiment the alpha-amylase is derived from Aspergillus kawachii and disclosed by Kaneko et al. J. Ferment. Bioeng. 81:292-298 (1996) “Molecular-cloning and determination of the nucleotide-sequence of a gene encoding an acid-stable alpha-amylase from Aspergillus kawachii.”; and further as EMBL:#AB008370.

The fungal alpha-amylase may also be a wild-type enzyme comprising a starch-binding domain (SBD) and an alpha-amylase catalytic domain (i.e., none-hybrid), or a variant thereof. In an embodiment the wild-type alpha-amylase is derived from a strain of Aspergillus kawachii.

An acid alpha-amylases may according to the invention be added in an amount of 0.001 to 10 AFAU/g DM, preferably from 0.01 to 5 AFAU/g DM, especially 0.3 to 2 AFAU/g DM or 0.001 to 1 FAU-F/g DM, preferably 0.01 to 1 FAU-F/g DM.

### Commercial Alpha-Amylase Products

Preferred commercial compositions comprising alpha-amylase include MYCOLASE™ from DSM (Gist Brocades), BAN™, TERMAMYL™ SC, FUNGAMYL™, LIQUOZYME™ X, LIQUOZYME™ SC and SAN™ SUPER, SAN™ EXTRA L (Novozymes A/S) and CLARASE™ L-40,000, DEX-LO™, SPEZYME™ FRED, SPEZYME™ AA, and SPEZYME™ DELTA AA (Genencor Int.), and the acid fungal alpha-amylase sold under the trade name SP288 (available from Novozymes A/S, Denmark).

### Carbohydrate-Source Generating Enzyme

The term “carbohydrate-source generating enzyme” includes glucoamylase (being glucose generators), beta-amylase and maltogenic amylase (being maltose generators) and also pullulanase and alpha-glucosidase. A carbohydrate-source generating enzyme is capable of producing a carbohydrate that can be used as an energy-source by the fermenting organism(s) in question, for instance, when used in a process of the invention for producing a fermentation product, such as ethanol. The generated carbohydrate may be converted directly or indirectly to the desired fermentation product, preferably ethanol. According to the invention a mixture of carbohydrate-source generating enzymes may be used. Especially contemplated mixtures are mixtures of at least a glucoamylase and an alpha-amylase, especially an acid amylase, even more preferred an acid fungal alpha-amylase. The ratio between acid fungal alpha-amylase activity (FAU-F) and glucoamylase activity (AGU) (i.e., FAU-F per AGU) may in an embodiment of the invention be between 0.1 and 100, in particular between 2 and 50, such as in the range from 10-40.

### Glucoamylase

A glucoamylase used according to the invention may be derived from any suitable source, e.g., derived from a microorganism or a plant. Preferred glucoamylases are of fungal or bacterial origin, selected from the group consisting of Aspergillus glucoamylases, in particular Aspergillus niger G1 or G2 glucoamylase (Boel et al. (1984), EMBO J. 3 (5), p. 1097-1102), or variants thereof, such as those disclosed in WO 92/00381, WO 00/04136 and WO 01/04273 (from Novozymes, Denmark); the A. awamori glucoamylase disclosed in WO 84/02921, Aspergillus oryzae glucoamylase (Agric. Biol. Chem. (1991), 55 (4), p. 941-949), or variants or fragments thereof. Other Aspergillus glucoamylase variants include variants with enhanced thermal stability: G137A and G139A (Chen et al. (1996), Prot. Eng. 9, 499-505); D257E and D293E/Q (Chen et al. (1995), Prot. Eng. 8, 575-582); N182 (Chen et al. (1994), Biochem. J. 301, 275-281); disulphide bonds, A246C (Fierobe et al. (1996), Biochemistry, 35, 8698-8704; and introduction of Pro residues in position A435 and S436 (Li et al. (1997), Protein Eng. 10, 1199-1204.

Other glucoamylases include Athelia rolfsii (previously denoted Corticium rolfsii) glucoamylase (see U.S. Pat. No. 4,727,026 and (Nagasaka, Y. et al. (1998) “Purification and properties of the raw-starch-degrading glucoamylases from Corticium rolfsii, Appl Microbiol Biotechnol 50:323-330), Talaromyces glucoamylases, in particular derived from Talaromyces emersonii (WO 99/28448), Talaromyces leycettanus (U.S. Pat. No. Re. 32,153), Talaromyces duponti, Talaromyces thermophilus (U.S. Pat. No. 4,587,215).

Bacterial glucoamylases contemplated include glucoamylases from the genus Clostridium, in particular C. thermoamylolyticum (EP 135,138), and C. thermohydrosulfuricum (WO 86/01831) and Trametes cingulata, Pachykytospora papyracea; and Leucopaxillus giganteus all disclosed in WO 2006/069289; or Peniophora rufomarginata disclosed in PCT/US2007/066618; or a mixture thereof. Also hybrid glucoamylase are contemplated according to the invention. Examples the hybrid glucoamylases disclosed in WO 2005/045018. Specific examples include the hybrid glucoamylase disclosed in Table 1 and 4 of Example 1 (which hybrids are hereby incorporated by reference).

Contemplated are also glucoamylases which exhibit a high identity to any of above mention glucoamylases, i.e., at least 70%, at least 75%, at least 80%, at least 85%, at least 90%, at least 95%, at least 96%, at least 97%, at least 98%, at least 99% or even 100% identity to the mature enzymes sequences mentioned above.

Commercially available compositions comprising glucoamylase include AMG 200L; AMG 300 L; SAN™ SUPER, SAN™ EXTRA L, SPIRIZYME™ PLUS, SPIRIZYME™ FUEL, SPIRIZYME™ B4U and AMG™ E (from Novozymes A/S); OPTIDEX™ 300 (from Genencor Int.); AMIGASE™ and AMIGASE™ PLUS (from DSM); G-ZYME™ G900, G-ZYME™ and G990 ZR (from Genencor Int.).

Glucoamylases may in an embodiment be added in an amount of 0.0001-20 AGU/g DM, preferably 0.001-10 AGU/g DM, especially between 0.01-5 AGU/g DM, such as 0.1-2 AGU/g DM.

### Biogas

The term “biogas” is according to the invention intended to mean the gas obtained in a conventional anaerobic fermentor, the primary digester. The main component of biogas is methane and the terms “biogas” and “methane” are in this application and claims used interchangeably.

### Primary Digester

The term “primary digester” is in this application and claims intended to mean the container wherein anaerobic fermentation takes place and biogas is produced.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

Various references are cited herein, the disclosures of which are incorporated by reference in their entireties. The present invention is further described by the following examples which should not be construed as limiting the scope of the invention.

### EXAMPLES

### Materials & Methods

### Cellulase Activity Using Filter Paper Assay (FPU Assay)

### 1. Source of Process

1.1 The process is disclosed in a document entitled “Measurement of Cellulase Activities” by Adney, B. and Baker, J. 1996. Laboratory Analytical Procedure, LAP-006, National Renewable Energy Laboratory (NREL). It is based on the IUPAC process for measuring cellulase activity (Ghose, T. K., Measurement of Cellulse Activities, Pure & Appl. Chem. 59, pp. 257-268, 1987.

### 2. Procedure

2.1 The process is carried out as described by Adney and Baker, 1996, supra, except for the use of a 96 well plates to read the absorbance values after color development, as described below.

### 2.2 Enzyme Assay Tubes:

2.2.1 A rolled filter paper strip (#1 Whatman; 1×6 cm; 50 mg) is added to the bottom of a test tube (13×100 mm).
2.2.2 To the tube is added 1.0 mL of 0.05 M Na-citrate buffer (pH 4.80).
2.2.3 The tubes containing filter paper and buffer are incubated 5 min. at 50° C. (±0.1° C.) in a circulating water bath.
2.2.4 Following incubation, 0.5 mL of enzyme dilution in citrate buffer is added to the tube. Enzyme dilutions are designed to produce values slightly above and below the target value of 2.0 mg glucose.
2.2.5 The tube contents are mixed by gently vortexing for 3 seconds.
2.2.6 After vortexing, the tubes are incubated for 60 mins. at 50° C. (±0.1° C.) in a circulating water bath.
2.2.7 Immediately following the 60 min. incubation, the tubes are removed from the water bath, and 3.0 mL of DNS reagent is added to each tube to stop the reaction. The tubes are vortexed 3 seconds to mix.

### 2.3 Blank and Controls

2.3.1 A reagent blank is prepared by adding 1.5 mL of citrate buffer to a test tube.
2.3.2 A substrate control is prepared by placing a rolled filter paper strip into the bottom of a test tube, and adding 1.5 mL of citrate buffer.
2.3.3 Enzyme controls are prepared for each enzyme dilution by mixing 1.0 mL of citrate buffer with 0.5 mL of the appropriate enzyme dilution.
2.3.4 The reagent blank, substrate control, and enzyme controls are assayed in the same manner as the enzyme assay tubes, and done along with them.

### 2.4 Glucose Standards

2.4.1 A 100 mL stock solution of glucose (10.0 mg/mL) is prepared, and 5 mL aliquots are frozen. Prior to use, aliquots are thawed and vortexed to mix.
2.4.2 Dilutions of the stock solution are made in citrate buffer as follows:
G1=1.0 mL stock+0.5 mL buffer=6.7 mg/mL=3.3 mg/0.5 mL
G2=0.75 mL stock+0.75 mL buffer=5.0 mg/mL=2.5 mg/0.5 mL
G3=0.5 mL stock+1.0 mL buffer=3.3 mg/mL=1.7 mg/0.5 mL
G4=0.2 mL stock+0.8 mL buffer=2.0 mg/mL=1.0 mg/0.5 mL
2.4.3 Glucose standard tubes are prepared by adding 0.5 mL of each dilution to 1.0 mL of citrate buffer.
2.4.4 The glucose standard tubes are assayed in the same manner as the enzyme assay tubes, and done along with them.

### 2.5 Color Development

2.5.1 Following the 60 min. incubation and addition of DNS, the tubes are all boiled together for 5 mins. in a water bath.
2.5.2 After boiling, they are immediately cooled in an ice/water bath.
2.5.3 When cool, the tubes are briefly vortexed, and the pulp is allowed to settle. Then each tube is diluted by adding 50 microL from the tube to 200 microL of ddH2O in a 96-well plate. Each well is mixed, and the absorbance is read at 540 nm.
2.6 Calculations (examples are given in the NREL document)
2.6.1 A glucose standard curve is prepared by graphing glucose concentration (mg/0.5 mL) for the four standards (G1-G4) vs. A540. This is fitted using a linear regression (Prism Software), and the equation for the line is used to determine the glucose produced for each of the enzyme assay tubes.
2.6.2 A plot of glucose produced (mg/0.5 mL) vs. total enzyme dilution is prepared, with the Y-axis (enzyme dilution) being on a log scale.
2.6.3 A line is drawn between the enzyme dilution that produced just above 2.0 mg glucose and the dilution that produced just below that. From this line, it is determined the enzyme dilution that would have produced exactly 2.0 mg of glucose.
2.6.4 The Filter Paper Units/mL (FPU/mL) are calculated as follows:
FPU/mL=0.37/enzyme dilution producing 2.0 mg glucose

### Xylose/Glucose Isomerase Assay (IGIU)

1 IGIU is the amount of enzyme which converts glucose to fructose at an initial rate of 1 micromole per minute at standard analytical conditions.

Standard Conditions:

Glucose concentration: 45% w/w

pH: 7.5

Temperature: 60° C.

Mg2+ concentration: 99 mg/l (1.0 g/l MgSO4*7 H2O)

Ca2+ concentration<2 ppm

Activator, SO2 concentration: 100 ppm (0.18 g/l Na2S2O5)

Buffer, Na2CO3, concentration: 2 mM Na2CO3

### Cellulytic Activity (EGU)

The cellulytic activity may be measured in endo-glucanase units (EGU), determined at pH 6.0 with carboxymethyl cellulose (CMC) as substrate.

A substrate solution is prepared, containing 34.0 g/l CMC (Hercules 7 LFD) in 0.1 M phosphate buffer at pH 6.0. The enzyme sample to be analyzed is dissolved in the same buffer. 5 ml substrate solution and 0.15 ml enzyme solution are mixed and transferred to a vibration viscosimeter (e.g. MIVI 3000 from Sofraser, France), thermostated at 40° C. for 30 minutes.

One EGU is defined as the amount of enzyme that reduces the viscosity to one half under these conditions. The amount of enzyme sample should be adjusted to provide 0.01-0.02 EGU/ml in the reaction mixture.

### Pectate Lyase Activity (APSU)

Pectate Lyase catalyses the formation of double bonds in polygalacturonic acid. The number of formed double bonds is determined by photometric measurement at 235 nm. One APSU (Alcalophile Pectate Lyase Unit) is defined as the amount of enzyme that produces C═C double bonds equivalent to 1 μmol unsaturated digalacturonic acid per minute under the standard conditions:

### Temperature: 37.0° C.±0.5° C.

pH: 10.00±0.05
Wavelength: 235 nm in a 1 cm cuvette
Incubation time: 10 min.

### Time of Measurement: 30 min.

Enzyme concentration range: 0.05-0.15 APSU/mL
Limit of quantification: 1.25 APSU/g
Range: [50; 150] mAPSU/mL

### Other Processes

Dry matter: Mettler Toledo HR 73 Halogen Moisture dryer
BRIX: RFM830 Digital refractometer from Bilingham & Stanley Ltd.
pH: WTW pH-meter
Milling: “coffee” grinder Bosch type KM13 (E nr: M KM 6003 FD 9512) for 2 minutes.
HPLC: Waters 717 Autosampler, Waters 515 Pump and a Waters 2414 Refractive index detector. A column type Bio-rad (Animex HPX-87 H 300-7.8 mm), Cat no. 125140 was used. Standards were used for glucose, maltose, maltotriose, xylose, and maltotetraose.

### Enzymes Used in the Examples:

A pectate lyase (EC 4.2.2.2) preparation derived from a Bacillus sp. is commercially available as BIOPREP® 3000 L (Novozymes A/S, Denmark) with an activity of 3000 APSU/g composition.

An endo-xylanase (EC 3.2.1.8) composition derived from Bacillus agaradhaerens is commercially available PULPZYME® HC (Novozymes A/S, Denmark).

A Cellulase composition A comprising acid cellulolytic enzymes derived from Trichoderma reesei, a GH61A polypeptide disclosed in WO2005/074656, and an Aspergillus oryzae beta-glucosidase (in the fusion protein disclosed in WO2008/057637). The cellulase composition A is disclosed in WO2008/151079. The cellulase composition A has an activity 180 FPU/g composition.

A cellulase composition B comprising alkaline endo-cellulase derived from Bacillus sp. is commercially available as CELLUCLEAN® 5.0 L (Novozymes A/S, Denmark) with an activity of 320000 ECU/g composition.

A ferulic acid esterase composition also comprising alkaline cellulase. The composition is derived from Humicola insolens and commercially available as NOVOZYM® 342 L (Novozymes A/S, Denmark) with an activity of 90 EGU/g

A mannanase (EC 3.2.1.25) composition comprising a mannanase with an activity of 40 MIUM/g composition.

### Example 1

A pre-testing of an alkaline enzyme system (E1) consisting of NOVOZYM® 342 L, PULPZYME® HC, CELLUCLEAN® 5.0 L and BIOPREP® 3000 L (All from Novozymes A/S, Denmark) was performed on pre-milled samples supplied from Nordic Sugar, Nakskov, Denmark, as follows:
1.10 g of sugar beet pulp material was suspended in 20 g water at 50° C.
2. pH was adjusted to 8 using 4 N NaOH.
3. To the time t=0, 0.05 g (50 μL) of each of the E1 enzyme products NOVOZYM® 342 L, PULPZYME® HC, CELLUCLEAN® 5.0 L and BIOPREP® 3000 L was added to the mix.
4. The reaction was carried out in a conical flask kept under stirring in a shaking table at 50° C.
5. After 10 minutes a Zero-sample was taken and frozen down for later assay. The samples are 2 mL. Samples are again taken at t=30 minutes, 60 minutes, 120 minutes and 240 minutes.
6. The assay was as follows; results are shown below in table 1:
   a. Centrifugation for 10 minutes at 14.000 RPM
   b. Measuring of degree ° Brix.
   c. Measuring of absorbance at 235 nm in a quarts cuvette.

TABLE 1
Reaction results during the hydrolysis
reaction of milled sugar beet material.

| Time | Absorbance | | | |
|---|---|---|---|---|
| (min) | (235 nm) | Δ A(235) | ° Brix | Δ Brix |
| 0 | 1.535 | 0 | 1.61 | 0 |
| 30 | 1.535 | 0 | 1.75 | 0.14 |
| 60 | 1.685 | 0.15 | 1.82 | 0.21 |
| 120 | 2.042 | 0.507 | 1.96 | 0.35 |
| 240 | 2.366 | 0.831 | 1.96 | 0.35 |
| 1080 | 3.35 | 1.815 | 2.44 | 0.83 |

Significant improved biogas production was found in a test system developed by Nordic Sugar and University of Hohenheim when compared to not pre-treated sugar beet pulp (not shown).

### Example 2

A two-step enzymatic production of hydrolysate based on pre-milled sugar beet pulp supplied from Nordic Sugar, Nakskov, Denmark, as follows:
1. The dry matter content of the beet pulp was measured using a HR 73 Halogen moisture analyzer to: 15.0% w/w.
2. 150 g of beet pulp was blended by hand into 300 mL of city water in each of two flasks.
3. pH was measured and adjusted to approx. pH=8.5. Approximately 1.5 mL 4 N NaOH was added to each flask. Stirring was with a powerful stirrer used at 150 rpm. No enzymes were added to flask no. 1.
4. Enzymes (E1) were added to flask no. 2. A dosage of 0.25% enzyme product of dry matter was used of each of the 4 enzyme products mentioned above in example 1. The dry matter content of the reaction mixtures was estimated based on the masses and the measurement of the pulp's dry matter content measured to 5.0%. Mass of dry matter: 150×5.01/100=22.5 (g dry matter used for dosage). This corresponded to 56.3 mg˜56.3/1.10˜50 μL, which was added.
5. pH and ° Brix was measured and the reactions were continued overnight. The measurements are shown in Table 2 below:

TABLE 2
pH and Brix data; a pH drop was detected
for both flasks due to demethylation.

| | | | ° BRIX (of |
|---|---|---|---|
| | | | supernatant |
| Date and time | Sample | pH | or filtrate) |
| Jan. 06, 2010 at 17:00 | Flask 1 | 6.35 (before | n.a. |
| | | adjustment). | |
| Jan. 06, 2010 at 17:00 | Flask 2 | 6.25 (before | n.a |
| | | adjustment). | |
| Jan. 06, 2010 at 17:45 | Flask 1 | 1.5 mL 4N NaOH | n.a. |
| | | was added: | |
| | | pH = 8.85 | |
| Jan. 06, 2010 at 17:45 | Flask 2 | 1.5 mL 4N NaOH | n.a |
| | | was added | |
| | | 1.5 mL 4N NaOH: | |
| | | pH = 8.40 | |
| Jan. 06, 2010 at 18:00 | Flask 1 | 8.30 | 1.49 |
| Jan. 06, 2010 at 18:00 | Flask 2 | 7.90 | 1.28 |
| Feb. 06, 2010 at 9:40 | Flask 1 | 5.14 | 1.62 |
| Feb. 06, 2010 at 9:40 | Flask 2 | 5.15 | 1.28 |
| Feb. 06, 2010 at 14:40 | Flask 1 | 5.07 | 1.62 |
| Feb. 06, 2010 at 14:40 | Flask 2 | 5.35 | 1.35 |
| Feb. 06, 2010 at 17:00 | Flask 1 | 4.72 | 1.35 (filtered) |
| Feb. 06, 2010 at 17:00 | Flask 2 | 5.94 | 1.21 (filtered) |

6. Methanol was detected in the reaction mixture after the enzymatic reaction (Flask no. 2) as shown by the HPLC result in table 3 below:

TABLE 3
HPLC-results after the alkaline enzymatic treatment

| | g/L | | | | | |
|---|---|---|---|---|---|---|
| | | | | Acetic | Lactic | % w/w |
| Sample | Glucose | Xylose | Arabinose | acid | acid | Methanol |
| Flask no. | 0.89 | 0.97 | 0 | 0.13 | 0.82 | 0.00 |
| 1 (Blind) | | | | | | |
| Flask no. | | | 0 | | 0.36 | 0.42-0.47 |
| 2 | | | | | | |

7. The pH drop shown in table 2 was utilized as a way to reduce pH in situ for use of the acidic saccharification enzyme system (E2) for both pectins and hemicelluloses. A dosage of 0.5% VISCOZYME® L (Novozymes A/S, Denmark) based on raw material dry matter, corresponding to 100 microliter VISCOZYME® L, was added.
8. Measurements of pH and ° Brix are shown in table 4.

TABLE 4
pH and Brix data during treatment with Viscozyme L.

| | | | ° BRIX (of |
|---|---|---|---|
| | | | supernatant |
| Date and time | Sample | pH | or filtrate) |
| Mar. 06, 2010 at 10:15 | Flask 1 | 4.04 | 3.61 |
| Mar. 06, 2010 at 10:15 | Flask 2 | 4.35 | 2.79 |
| Apr. 06, 2010 at 10:15 | Flask 1 | 3.97 | 3.54 |
| Apr. 06, 2010 at 10:15 | Flask 2 | 4.37 | 3.20 |

9. A centrifugation index (estimate for % yield of solubilised material) was calculated; results are shown in table 5 below:

TABLE 5
Centrifugation indices.

| | Centrifugation index based |
|---|---|
| | on ° Brix values and total dry |
| Sample | matter in the reaction mixture |
| Flask 1 | 0.70 |
| Flask 2 (enzym liquefaction) | 0.64 |

10. HPLC analyses were carried out on the supernatants, the results are shown in table 6 below:

TABLE 6
HPLC-results after the treatment with VISCOZYME ® L
(nd*: not determined).

| | | c) | | | | | |
|---|---|---|---|---|---|---|---|
| | | g/L | | | | | w/w % |
| a) | b) | Glu- | Xy- | Arabi- | Acetic | Lactic | Meth- |
| Sample | Vial | cose | lose | nose | acid | acid | anol |
| Flask no. 1 | 33 | 0.36 | 0.41 | 2.56 | nd*) | 3.39 | 0.22 |
| (Blind) | | | | | | | |
| Flask no. 2 | 34 | 0.44 | 0.40 | 0.85 | nd*) | 2.15 | 0.26 |

### Example 3

1. The dry matter content of the beet pulp was measured using a HR 73 Halogen moisture analyzer to: 26.7% w/w.
2. 94 g of beet pulp was blended by hand into 406 mL of city water.
3. pH was measured and adjusted to pH=9 using NaOH using 4 N NaOH. Stirring with a powerful stirrer (150 rpm. The temperature was 50.0° C. during the reaction.
4. Enzymes (E1) were added to all flasks. A dosage of 0.25% enzyme product of dry matter was used of each of the 4 enzyme products mentioned in example 1.
5. Hydrolysis was carried out for 23 hours.
6. VISCOZYME® L (E2) in 0.5% on dry matter was added when pH was 5.12. Hydrolysis was running for additionally 78 h.
7. ° Brix and pH were measured over the complete trial; results in table 7 (below).
8. Samples were drawn for HPLC and the results are shown in table 8 (below).
9. A biogas trial was carried according to VDI-RICHTLINIEN, VDI 4630 (2006) title: Fermentation of organic materials. Characterisation of the substrate, sampling collection of material data, fermentation tests. The results of the test are shown in table 9 below.

TABLE 7
Data measured over the reaction.

| | Total | | |
|---|---|---|---|
| | reaction | | |
| Event | time, h | pH | ° Brix |
| Initial | 0.0 | 9 | N.A. |
| Reaction with E1 ended | 23 | 6.41 | 0.99 |
| Reaction with E2 | 41 | 5.12 | 1.2 |
| Reaction with E2 | 50 | 4.42 | 2.17 |
| Reaction with E2 ended | 101 | 3.99 | 2.92 |

TABLE 8
HPLC results

| | Glu- | Xy- | Arabi- | Acetic | Lactic | Meth- |
|---|---|---|---|---|---|---|
| Galacturonic | cose, | lose, | nose, | acid, | acid, | anol, |
| acid, g/L | g/L | g/L | g/L | g/L | g/L | % w/w |
| Not measured | 0.27 | 0.49 | 1.18 | 1.42 | 1.97 | 0.48 |

TABLE 9
Biogas yields according to VDI 4630 (2006).

| Parameter | Result |
|---|---|
| Sample amount | 1.28 g |
| Volume gas developed over 40 hours | 23 mL |
| Methane content | 50% |
| N m³ methane/kg Brix, 40 h | 0.37 |

### Example 4

In this example is illustrated how a pre-treatment is carried out in pilot plant. The overall process layout of the complete biogas process is shown in FIG. 1.
1. 44.5 kg pulp was wet milled in 200 kg water held under recirculation in the liquefaction tank at 45° C. Small portions of fresh pulp were added to the funnel of the mill as shown in FIG. 1. The mill was a toothed colloid mill (Romaco, FrymaKoruma Type MZ80).
2. The first 10 kg pulp was added together with about 60 g soda powder, which was also added in small portions during the wet milling process.
3. pH was held about 8.5 and it was checked regularly over the time where the pulp was added (about 15 minutes).
4. 129 g of Enzyme 1 (BIOPREP® 3000L, PULPZYME® HC and NOVOZYM® 342 L) was added to the recirculating slurry, in the tank.
5. The rest of the pulp, about 40 kg was added through the Fryma tract and pH was maintained at 8-8.5 by addition of about 200 g soda powder. This procedure lasted 1.5 hour. The reaction temperature was re-adjusted to 45° C.
6. When all pulp was mixed into the tank the reaction continued with intermediate intervals of 1 hour reaction for stirring, and Fryma-wet millings for 30 minutes.
7. The liquefaction reaction was followed with measurements of ° Brix, pH, HPLC-analyses; results are shown below in tables 10 and 11.
8. The reaction was finished in 10% hours, when pH levelled out at 5.5-6.0.
9. The second hydrolysis was initiated by addition of 64.5 g Enzyme 2 (VISCOZYME® L).
10. The Fryma-mill was used during the first 30 minutes and measurements of ° Brix, pH, and HPLC was carried out (Table 10 and table 11).
11. The total reaction time during this hydrolysis step was hours. For the “Beet pulp saccharified 19% h” the sum of the values shown above account for ca. 70% of the Brix-value.

TABLE 10
Data measured over the reactions.

| | | Temperature, | | % | |
|---|---|---|---|---|---|
| Time, h | pH | ° C. | ° Brix | sludge | Remarks |
| 0 | 4.3 | 45 | n.a. | n.a. | Initial |
| | | | | | reaction |
| | | | | | with E-1 |
| 0.5 | 7.6 | 46 | 1.4 | ca. 50 | Milling |
| | | | | | finished |
| 9.75 | 6.1 | 44 | 1.8 | 52 | |
| 10 | 6.1 | 46 | 1.8 | 52 | E-2 was |
| | | | | | added |
| 24.3 | 4 | 46 | 2.5 | 35 | Thin like |
| | | | | | juice |

TABLE 11A
HPLC results.

| | Oligo- | | | | |
|---|---|---|---|---|---|
| | Saccharides, | Galacturonic | Glucose | Xylose | Arabinose |
| Sample | g/l | acid, g/l | g/l | g/l | g/l |
| Beet pulp | 5.8 | 0.0 | 0.0 | 0.0 | 0.0 |
| liquefied | | | | | |
| 10¼ h | | | | | |
| Beet pulp | 10.4 | 0.4 | 0.2 | 0.1 | 0.3 |
| saccharified | | | | | |
| 19¼ h | | | | | |

TABLE 11B
HPLC results.

| | Lactic acid | Acetic acid | Methanol | Ethanol |
|---|---|---|---|---|
| Sample | g/l | g/l | g/l | g/l |
| Beet pulp | 1.1 | 0.5 | 0.2 | 0.5 |
| liquefied | | | | |
| 10¼ h | | | | |
| Beet pulp | 3.6 | 1.8 | 0.4 | 0.4 |
| saccharified | | | | |
| 19¼ h | | | | |

### Example 5

Pre-treatments were carried out with Fryma milling of ensilaged sugar beet pulp from Nordic Sugar. Biogas trials were carried out in the laboratory in order to demonstrate improvements of biogas yield and shorter fermentation as a result of the enzymatic pre-treatment process. The reaction parameters for the two pre-treatment trials are shown in table 12.

TABLE 12
Reaction parameters for two pilot plant trials.

| | | | | | | | Dosage of | |
|---|---|---|---|---|---|---|---|---|
| | | | | Dosage of E-mix | | | Viscozyme L | |
| | | | | in weight % of | Reaction | | in weight % of | Reaction |
| | | Mass or volume | | the mass of | time with | | the mass of | time with |
| Trial | Alkali used | of alkali used | | pulp dry matter | E-mix | | pulp dry matter | Viscozyme L |
| A | soda powder | 860 | g | 1.0% (125 g) | 24.3 | h | 0.5% (62.5 g) | 22.0 h |
| | (Na₂CO₃) | | | | | | | |
| B | 27% NaOH | 1.2 | Litre | 1.5% (188 g) | 19 | h | 0.5% (62.5 g) | 28.0 h |

TABLE 13
Final measurements for two pilot plant trials.

| Trial | pH at end | ° Brix | % sludge |
|---|---|---|---|
| A | 4.4 | 3.6 | 35 |
| B | 4.5 | 3.4 | 45 |

TABLE 14A
HPLC results for two pilot plant trials at the end.

| Trial | Oligosaccharides | Galacturonic | Glucose | Xyloxe | Galactose | Arabinose | Acetic acid |
|---|---|---|---|---|---|---|---|
| End-products | (g/l) | acid (g/l) | (g/l) | (g/l) | (g/l) | (g/l) | (g/l) |
| A | 14.8 | 2.4 | 0.1 | 0.2 | 0.2 | 1.2 | 5.7 |
| B | 11.7 | 2.5 | 0.1 | 0.9 | 1.7 | 1.2 | 5.4 |

TABLE 14B
HPLC results for two pilot plant trials at the end (cont'd).

| | | Lactic | | | | |
|---|---|---|---|---|---|---|
| Trial | Glycerol | acid | Methanol | Ethanol | | |
| End-products | (w/w %) | (g/l) | (w/w %) | (w/w %) | Sum | °Brix |
| A | 0.03 | 1.6 | 0.06 | 0.07 | 26.3 | 3.6 |
| B | 0.02 | 1.2 | 0.04 | 0.04 | 24.8 | 3.4 |

### Wet Milling, Liquefaction and Hydrolysis in Pilot Plant (Trial A):

This pilot plant trial was carried out on ensilaged sugar beet pulp. Soda and the enzyme-system E-1 was added during the milling process. Biogas production in laboratory scale was carried out on the liquefied and saccharified pulp.
1. 200 kg water and 50 kg ensilaged pulp was wet-milled in recirculating water at about 45° C. by addition of small portions of pulp to the funnel as shown for the liquefaction step in FIG. 1. The first 10 kg was added together with about 60 g soda powder (Na₂CO₃) which was also added in small portions during the wet-milling process. pH was held at 8.5 and it was checked regularly over the blending period (10 minutes).
2. At this point of time 125 g of E-mix was added to the recirculating slurry, in the tank.
3. The rest of the pulp, about 40 kg was added directly to the Fryma tract and pH was maintained at 8-8.5 by addition of about 600 g soda powder. This complete procedure lasted 60 minutes. The reaction temperature was re-adjusted to 45° C. using cooling on the tank mantel.
4. When all pulp had been mixed into the reaction tank the reaction continued with intermediate intervals of one hour reaction for stirring, and wet millings for 30 minutes. The liquefaction reaction was followed with measurements of ° Brix, pH and HPLC.
5. The reaction was finished in 24.3 hours, when pH leveled out at 6.13.
6. The second hydrolysis was initiated by addition of 62.5 g Viscozyme®L.
7. The wet milling was carried out again for 30 minutes and samples were drawn for measurements of ° Brix, pH, and HPLC.
8. The reaction was finished the next morning after totally 46.3 hours processing

### Wet Milling, Liquefaction and Hydrolysis in Pilot Plant (Trial B):

1. In total 50 kg ensilaged pulp from Nordic Sugar was wet milled in 193 liter water held under recirculation at about 45° C. for the whole trial.
2. Small portions of pulp were added to the funnel of the mill as shown for the liquefaction step in FIG. 1. The first 10 kg was added together with about 0.3 L of 27% NaOH which was also added in small portions during the wet milling process that lasted 10 minutes.
3. pH was kept at 8.5 from the start and ongoing.
4. Hereafter 188 g of E-mix was added to the recirculating slurry, in the tank.
5. The rest of the pulp, about 40 kg was added through the tract of the mill, and pH was still maintained at 8.5. 0.9 L of 27% NaOH was added over 40 minutes. The temperature was is re-adjusted to 45° C. using cooling on the tank mantel.
6. The reaction continued with intermediate intervals of 1 hour reaction for stirring, and Fryma-wet millings of 30 minutes milling.
7. The liquefaction reaction was followed with measurements of ° Brix, pH, HPLC.
8. The reaction was finished in 19 hours, when pH leveled out at 5.8.
9. The second hydrolysis was initiated by addition of 62.5 g Viscozyme®L.
10. The wet milling was carried out again for 30 minutes and samples were drawn for measurements of ° Brix, pH, and HPLC.
11. The reaction was finished the next morning after totally 46.9 hours processing.

### Method for Biogas Trials:

### Description of the Mesophilic Fermentation Tests:

In order to assess the effect of different pre-treatments and on the anaerobic biodegradability series of mesophilic (37° C.±2° C.) short-term batch tests were performed on lab-scale biogas reactors. The reactor set-up consisted of a 1.0 L Erlenmeyer flask, placed in a thermostated water bath at 37° C. and connected to a biogas column. At the start of each test series, the reactors (4 to 6 reactors per test series) were seeded with the same amount of fresh thermophilic anaerobic sludge. In this case the seeding sludge originated from a full-scale UASB reactor of a potato-processing factory.

The feeding of the seeding sludge with a certain amount of the different beet pulp substrates was done manually. After the feeding and pH measurement of the mixed liquors, each reactor was connected to a column to follow the biogas production on a daily basis.

At the end of a digestion cycle (about 1, 2 or 3 weeks per feeding cycle), samples were taken to analyze the residual volatile fatty acids and soluble COD concentrations. The methane concentration of the produced biogas was also determined.

For each treatment, three to four successive feeding cycles using the same beet pulp substrate were performed. In one trial sucrose was tested as a positive control. Standard analyses on the products were performed as described in the art. In short the total solid content (TS %) was determined by drying at 105° C. until no further weight change occurred. Ash was determined in a muffle furnace by heating the sample to 600° C. in a crucible until no further weight change occurred. Volatile solid (VS %) was calculated by subtracting total solids with ash content.

Chemical oxygen demand (COD) was measured by the potassium dichromate method as described by Greenberg et al. in Standard Methods for the examination of water and wastewater. 18th Edition, 1992, p. 5-7. The products with the analytical characteristics shown in table 15 were tested for yield of biogas.

TABLE 15
Products tested for biogas production in the fermentation trials.

| | TS | Ash | VS | COD | COD/ |
|---|---|---|---|---|---|
| Products tested | % | % | % | (g/L) | VS |
| Sucrose | 100 | 0 | 100 | — | — |
| Ensilaged Sugar | 25.13 | 1.81 | 23.32 | — | — |
| Beet Pulp (ESBP) | | | | | |
| Wet Milled Sugar | 11.89 | 0.72 | 11.17 | 74 | 0.66 |
| Beet Pulp (WMSBP) | | | | | |
| Hydrolysed sugar | 4.37 | 0.76 | 3.61 | 57.1 | 1.58 |
| beet pulp (Trial A) | | | | | |
| Hydrolysed sugar | 4.46 | 0.94 | 3.52 | 55.8 | 1.59 |
| beet pulp (Trial B) | | | | | |

To analyze the methane formation, samples of the bottle headspace volumes were taken with a Hamilltion gas syringe and subjected to GC analysis on a Varian 3900 gas chromatograph with a PoraPLOT Q (10 μm) 25 m×0.32 mm fused silica separation column (Varian, Agilent Technologies, USA).

### The Fermentation Trials:

Batch tests in two or three replicas fermentation trials were carried out at a load of 2 g dry matter per feeding cycle. Inoculation of each reactor was with 150 g of wet granular mesophilic sludge from a potato-processing plant. The TS % of the seed sludge was 17.2% w/w. The ash content of the seed sludge was 7.4% w/w; thus in each reactor 14.7 g volatile solid (DM−ash) was added.

After each cycle 100 mL effluent were removed, sieved and solids were returned to each reactor. C-source+tap-water to a total feeding volume of 100 mL were added. In table 16 the dosages and quality of the substrates for each cycle are shown. Standard parameters like liter cumulative biogas production, CH₄-production, COD reduction, soluble COD before and after each cycle. Control of pH, NH₄⁺ and total N were checked.

TABLE 16
Dosage and quality of the substrates for each cycle.

| | Type of substrate | | | | |
|---|---|---|---|---|---|
| | | | Wet | Hydrolyzed | Hydrolyzed |
| | | Ensilaged | milled | pulp | pulp |
| | Sucrose | pulp | pulp | (Trial A) | (Trial B) |
| Amount/ | 2 | 8.58 g | 17.9 g | 55.4 g | 56.8 g |
| feeding | | | | | |
| (g WW) | | | | | |
| g VS/ | | 2 | 2 | 2 | 2 |
| feeding | | | | | |
| g COD/ | 2 | | 1.32 | 3.16 | 3.17 |
| feeding | | | | | |

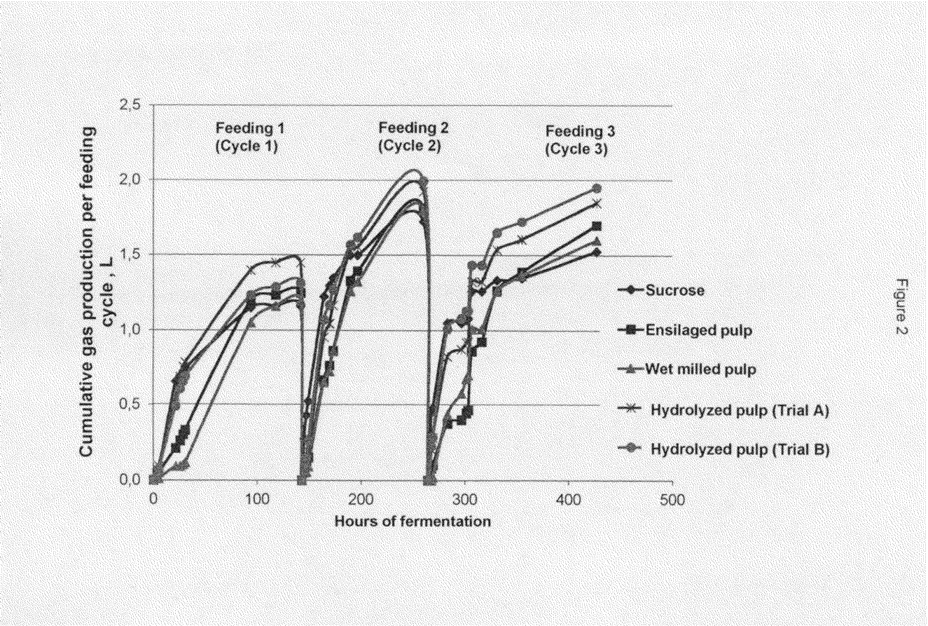

In FIG. 2 the evolution of the cumulative biogas production during each cycle is illustrated. In cycle 2 and 3 the rate of the biogas production was higher for the enzyme hydrolyzed pulps.

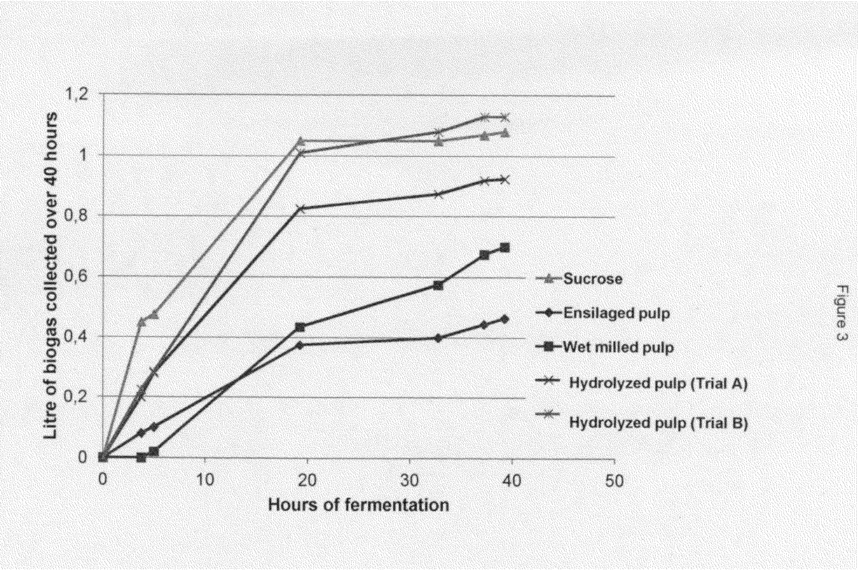

FIG. 3 shows that significant more biogas at higher production rate could be obtained using the enzymatically hydrolyzed pulps. The pulp described above under Trial B followed the rate of fermentation for sucrose. The difference of the performance of the two hydrolysates is assumed to be due to mainly the contents of xylose and galactose as indicated in table 4 as a result of the use of a higher enzyme dosage (table 12).

pH after all fermentation cycles were in the interval pH=6.9-7.3. COD per feeding was reduced 87% (average for all the trials) over the fermentations.

Table 17 shows the average biogas and methane production and content in the collected biogas from the three cycles run in 427 hours (FIG. 2).

TABLE 17
Average production of biogas and methane, and content
in the biogas collected during the three cycles.

| | Average | Methane | Methane | Standard |
|---|---|---|---|---|
| | NL biogas/ | yield, | content | deviation |
| | kg VS | NL/kg | in the | on methane |
| | added | VS | biogas % | content |
| Sucrose | 647 | 452 | 70 | 2 |
| Ensilaged pulp | 699 | 497 | 71 | 2 |
| Wet milled pulp | 675 | 467 | 69 | 1 |
| Hydrolyzed pulp | 766 | 556 | 72 | 2 |
| (Trial A) | | | | |
| Hydrolyzed pulp | 771 | 549 | 71 | 2 |
| (Trial B) | | | | |

### Example 6

An illustration of the advantage of using the two step process as illustrated in FIG. 1 was made in the following trial in order to show the advantage over a single process using a multicomponent cell wall degrading enzyme.

In a preferred embodiment of our claim 1 the liquefaction is facilitated with Enzyme 1 in a dosage of 1% of beet pulp total solids using pH=8.5 at 50° C. from start. After about 12 hours pH dropped to 5-6 and Enzyme 2 in the dosage 0.5% of pulp total solids is added for saccharification over 18/20 hours.

Enzyme 1 included pectate lyase/pectin methyl esterase and hemicellulases. Enzyme 2 included endo-pectinase, pectin methyl esterase and polygalacturonase. This system worked fine on fresh pulp as well as on ensilaged pulps and produced a liquid hydrolysate that within about 48 hours could be fully digested to biogas.

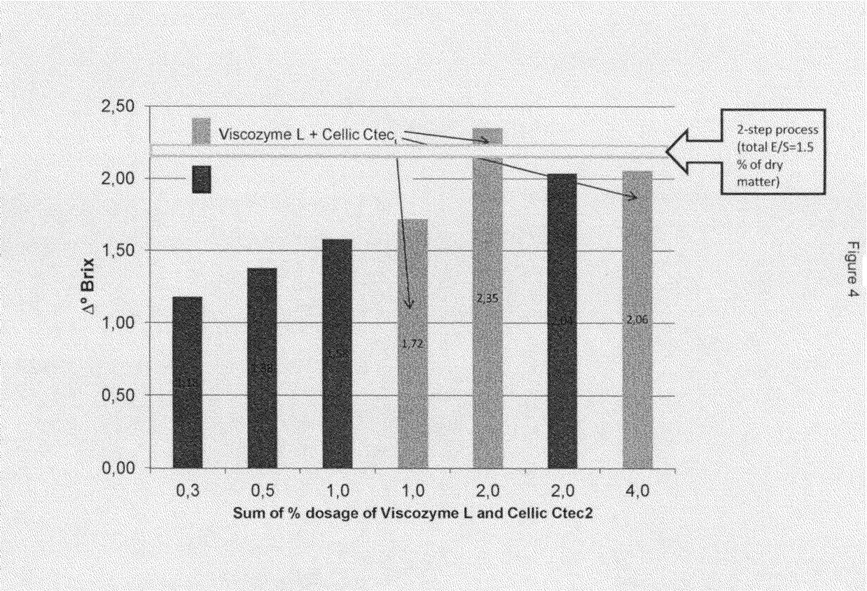

To simulate the performance of beet pulp using only acidic enzymes, Enzyme 2+Cellic®CTec2 (Novozymes A/S) at initial pH 4.5 were tested without prior liquefaction by an alkaline enzyme system (Enzyme 1). When the alkaline system was excluded, a four times higher dosage of Enzyme 2+Cellic CTec2 was necessary in order to obtain similar hydrolytic effect as could be obtained in the 2-step process, where the initiating swelling of the pulp with the alkaline enzyme system facilitated the overall hydrolysis. This result is illustrated by increase of soluble dry matter as measured by ° Brix and shown in FIG. 4. Also HPLC results indicated an insufficient conversion of the pulp (data not shown).

### Short Conclusion (all Examples):

The laboratory biogas trials illustrated the advantages of the 2-step enzymatic process of the invention. Improvements of biogas yield and shorter biogas fermentation were both demonstrated as compared to a single-step process.

1. A biogas production process with two enzymatic pre-treatments, said process comprising the steps of:
(a) providing a slurry comprising a lignocellulose- and pectin-containing material, water and one or more enzyme (FIG. 1; enzyme 1), including, at least one pectate lyase, pectin lyase and/or pectin methylesterase;
(b) allowing the one or more enzyme to catalyze the degradation of the material at a suitable temperature and a starting pH, wherein the pH drops to below 7 over time; and
(c) adding one or more additional enzyme (FIG. 1; enzyme 2) and allowing the one or more additional enzyme to catalyze the degradation of the material further at a suitable temperature and pH; and
(d) adding the enzyme-degraded material to a biogas digester tank at a suitable rate and ratio to effectively convert the material to biogas in the digester.

2. The process of claim 1, wherein the one or more enzyme or additional enzyme is selected from the group consisting of an amylolytic enzyme, a lipolytic enzyme, a proteolytic enzyme, a cellulolytic enzyme, an oxidoreductase and a plant cell-wall degrading enzyme.

3. The process of claim 2, wherein the one or more enzyme or additional enzyme is selected from the group consisting of aminopeptidase, alpha-amylase, amyloglucosidase, arabinofuranosidase, arabinoxylanase, beta-glucanase, carbohydrase, carboxypeptidase, catalase, cellobiohydrolase, cellulase, chitinase, cutinase, cyclodextrin glycosyltransferase, ferulic acid esterase, deoxyribonuclease, endo-cellulase, endo-glucanase, endo-xylanase, esterase, galactosidase, beta-galactosidase, glucoamylase, glucose oxidase, glucosidase, haloperoxidase, hemicellulase, invertase, isomerase, laccase, ligase, lipase, lyase, mannanase, mannosidase, oxidase, pectate lyase, pectin lyase, pectin trans-eliminase, pectin ethylesterase, pectin methylesterase, pectinolytic enzyme, peroxidase, protease, phytase, phenoloxidase, polygalacturonase, polyphenoloxidase, proteolytic enzyme, rhamnogalacturonan lyase, rhamnoglucanase, rhamnogalacturonase, ribonuclease, SPS-ase, transferase, transglutaminase, xylanase, xyloglucanase.

4. The process of claim 1, wherein the material or the slurry is homogenized; preferably by milling, wet-milling, grinding or wet-grinding prior to or during step (b) and/or step (c).

5. The process of claim 4, wherein a base is added to the material or the slurry prior to or while it is being homogenized; preferably the base is NaOH, Na₂CO₃, NaHCO₃, Ca(OH)₂, lime hydrate, ammonia and/or KOH.

6. The process of claim 1, wherein the starting pH in step (b) is between 7 and 12, such as from 7.6 to 10; preferably from 8 to 10, or from 8 to 9, preferably around pH 8.5.

7. The process of claim 1, wherein the content of the material in the slurry is adjusted by continuous or stepwise addition of material to the slurry during step (b) and/or step (c).

8. The process of claim 1, wherein the material constitutes above 2.5 wt-% DM, preferably above 5 wt-% DM, preferably above 10 wt-% DM, preferably above 15 wt-% DM, preferably above 20 wt-% DM, more preferably above 25 wt-% DM of the slurry of step a).

9. The process of claim 1, wherein step (c) is carried out at a pH in the range from 3 to 7; preferably from 4 to 6; most preferably at a pH value around 5.

10. The process of claim 1, wherein step (b) and/or (c) is carried out at a temperature in the range from 20-70° C., preferably 30-60° C., and more preferably 40-50° C.

11. The process of claim 1, wherein a solids separation step is performed after step (b) but before step (c) to purge not-solubilized solids (FIG. 1) and optionally feed them back into step (a) of the process.

12. The process of claim 1, wherein a solids separation step is performed after step (c) but before step (d) to purge not-solubilized solids (FIG. 1) and optionally feed them back into step (a) or (c) of the process.

13. The process of claim 1, wherein the material prior to step (a) has been subjected to a microwave and/or an ultrasonic irradiation treatment.

14. The process of claim 1, wherein the material has been chemically, mechanically and/or biologically pre-treated prior to step (a).

15. The process of claim 1, wherein the material comprises or is derived from potato pulp, sweet potato pulp, cassava pulp, sugar beet pulp, apple pulp, pear pulp, banana pulp, orange pomace, grape pomace, lemon pulp, pineapple pulp as well as waste residue from carrots, cereal straw, wheat straw, palm fronds, palm fruits, empty palm fruit bunches, palm residues, switch grass, miscanthus, rice hulls, municipal solid waste, industrial organic waste, office paper, bagasse of sugar cane or mixtures thereof.
